(19) United States
(12) Patent Application Publication
(10) Pub. No.: US 2002/0098216 A1
(43) Pub. Date: Jul. 25, 2002

(54) Illuminable lipstick applicator

(76) Inventor: Craig P. Nadel, Montville, NJ (US)

Correspondence Address: COHEN, PONTANI, LIEBERMAN & PAVANE, Suite 1210, 551 Fifth Avenue, New York, NY, 10176, US

(21) Appl. No.: 09/956,672

(22) Filed: Sep. 20, 2001

Related U.S. Application Data
(60) Provisional application No. 60/233,982, filed on Sep. 20, 2000.

(51) Int. Cl. A61K 7/00; B43K 23/00

(52) U.S. Cl. 424/401; 401/88

(57) ABSTRACT

A lipstick applicator includes a housing part having a bullet-shaped mass of lipstick with a fixed end mounted in the housing part and projecting outward to a free end remote from the housing part. The lipstick is formed of a light diffusing material, that is at least partly transmissive to light, and a selectively-actuable light source such as an LED is mounted in the housing part so that light emitted therefrom enters the fixed end of the mass and is diffused or transmitted along and within the mass to thereby internally illuminate the lipstick mass.

### RELATED APPLICATION

This application claims priority from U.S. Provisional Patent Application Serial No. 60/233,982 , which was filed on Sep. 20, 2000.

### BACKGROUND OF THE INVENTION

1. Field of the Invention

The present invention is directed to internally illuminated devices, and more particularly to a selectively or operatively illuminable applicator for lipstick, lip gloss, glitter, balm or other cosmetic materials that are user-applied and take the general form of an elongated, typically shaped mass of material.

The invention is herein described, by way of illustration, in embodiments providing a lipstick applicator, but is equally applicable to other, related uses—such for example to apply lip gloss or lip balm or other materials to a user's body—with little or no modification. Indeed, those skilled in the art will recognize that the inventive device and arrangement as herein disclosed may be additionally modified or utilized to apply non-cosmetic materials, such as crayon-type markers and stick-form adhesives and the like, to inanimate work surfaces.

2. Description of Related Art

Lipstick applicators, as heretofore known and readily available, are generally configured as elongated two-piece cylindrical housings in which the two mating housing parts are manually separable to reveal an elongated mass or “bullet” of lipstick secured to and projecting outward from one of the housing parts. The bullet-carrying or first housing part is selectively manipulable by the user to position and move the free or outward end of the bullet against and along the user's lips to apply the lipstick from the bullet to the lips. The second housing part forms a cover of the housing which protects the bullet and keeps it from drying out.

### SUMMARY OF THE INVENTION

A lipstick applicator in accordance with the present invention retains this general configuration or form and its operative utility and functionality. In addition, however, the invention provides structure and elements that illuminate the bullet in response to one or more various alternative forms of activation.

In accordance with the invention, a mass of light diffusing material has a fixed end mounted in a first housing part and projecting from the fixed end to a free end remote from this housing part. The material is applicable to a surface by moving contact with the surface, and is preferably in the form of a bullet. The first housing part further includes a light source mounted in the first housing part so that light emitted therefrom enters the fixed end of the mass, as well as a power source and a switch. In preferred forms of the invention, the light source includes a light emitting diode (LED), such as one operable to generate a red or other color or uncolored light, and the power source may comprise one or a plurality of conventional button-type or otherwise miniaturized batteries to power the LED.

When the LED is activated, in accordance with the invention the emitted light illuminates the bullet internally. Toward that end, the lipstick material of which the bullet is formed is transmissive to an extent sufficient to permit the light from the LED to diffuse through and reach the surface of the bullet, preferably along its entire length, so that the internally illuminated lipstick is visible to the user. This functionality may be implemented in any of various ways within the scope and contemplation of the invention. For example, the material may be translucent, or it may be relatively transparent and be filled with reflective particles or “glitter” which reflect and diffuse the light throughout the lipstick. To provide or enhance illumination of the bullet, it may include a fluorescing or phosphorescing or other light emitting or enhancing additive. In this case the LED may be selected to transmit at higher frequencies including the ultraviolet range in order to stimulate the phosphors to emit predetermined visible wavelengths.

In one implementation, the fixed end of the bullet is located immediately adjacent, or at least closely proximate, the LED, so that when the LED is activated the resulting light impinges on and is received by the fixed end of the bullet and is transmitted and diffused down and along the length of the bullet so as to impart a user-visible “glow” or illumination to the surface of the bullet. Transmission of light from the illuminated LED to the fixed end of the bullet may be enhanced or facilitated by placing a lens or other light-conducting surface—for example a circular collector plate—against or in substantial contact with the fixed end, between the end and the adjacent LED. Alternatively or additionally, a reflector may be provided around the base of the LED in order to direct light toward the bullet.

In another contemplated implementation, light transmitted from the LED along the bullet may be further enhanced by forming the bullet around (or otherwise providing in such an arrangement) a thin elongated rod or tube of light-conductive material—such as a plastic or fiber optic tube—that extends from the LED into the bullet, for example substantially centrally within and along the bullet. This arrangement will also permit an area of enhanced illumination at the free end of the projecting bullet. The light-conduction rod may also be connected, at one end, to a plate-like lens or concentrator or collector or element (as described above) disposed between the LED and bullet and preferably abutting the anchored end of the bullet. Other configurations for directing light from the LED into the light-conduction rod may also be employed, such as mounting the LED in a partially-surrounding cup or bowl (to which the rod is attached) to maximize the capture of light from the LED and direct the captured light into the rod.

The rod, instead of having a single, constant diameter, may in modified implementations bear one or more radially-extending projections along its length for carrying light closer to the surface of the bullet at predetermined locations, thereby providing increased illumination at those locations. In this manner seemingly random or design-evident patterns of illumination, as seen by a user, may be defined in the bullet.

The invention contemplates that the LED may be activated to emit light into the bullet in any one or more of various ways, only a representative sampling of which are herein described by way of illustrative example. In one embodiment, a spring-loaded push-to-operate switch selectively actuable by the user is located on the bullet-carrying housing part—preferably on the housing part periphery at the position at which the user typically grasps the housing part to manipulate and thereby apply lipstick from the bullet to the user's lips. In this way, the bullet can be readily illuminated by the user when holding the bullet-carrying housing part and/or when applying lipstick to the lips, as for example by applying inwardly-directed pressure to a deformable or movable section of the housing wall or against an activating button of the switch disposed in and/or through the wall.

In another contemplated embodiment, or as an addition to another actuation implementation, a pressure-sensitive switch may be incorporated within the housing so that, when the bullet is pressed against the user's lips to apply lipstick to the lips, the side-to-side (lateral) or end-exerted (longitudinal) pressure on the bullet activates the pressure-sensitive switch to cause the bullet to illuminate or “glow”.

In yet another implementation, or as a further addition to another of the described or contemplated embodiments, a switch actuated by a change in orientation of the bullet-carrying housing—such as what is sometimes referred to as a “shake-to-actuate” switch—can be provided so that the bullet is illuminated randomly or otherwise in response to movement or shaking of the housing. Such an arrangement may optionally be augmented by suitable structure or elements to prevent motion-initiated activation of the LED when the housing is closed, or to otherwise disable this feature so that the LED will not be unintentionally activated during periods of storage or nonuse, such as when the closed housing is being carried or transported on a user's person or within a handbag or carrying case.

The inventive applicator may also be provided with timing circuitry or the like so that, whenever activated, the LED will continue to emit light (by continued application of power thereto) for a predetermined or randomly (or otherwise varying) period of time beyond the termination of the cause of the initial activation. In such an implementation—or indeed in any other(s) of the herein described or contemplated embodiments—it is also within the intended scope of the invention that the LED, instead of continuously emitting light in response to activation, may flash or pulse at a single or a varying rate.

It is also intended that the second housing part, i.e. the removable “cover” of the housing—may be formed in whole or part of a transparent or translucent material to permit the bullet (or parts thereof), when illuminated, to be viewed through the transparent or translucent housing part. Similarly, the housing cover may be “patterned”, as for example with design or pattern-forming grooves or indentations or relief patterns, which will receive illumination from the bullet and thereby define an illuminated pattern on the housing cover which the user can view.

It should also be understood that, although the herein described and illustrated structure does not expressly include or depict a mechanism or elements, as is common and conventional in such applicators, for selectively adjusting and varying the projection of the bullet outwardly from the housing to which it is retained, such additional adjustment mechanism or elements may of course be included as a matter of design choice in any applicator constructed in accordance with the invention.

Other objects and features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims. It should be further understood that the drawings are not necessarily drawn to scale and that, unless otherwise indicated, they are merely intended to conceptually illustrate the structures and procedures described herein.

### BRIEF DESCRIPTION OF THE DRAWINGS

In the drawings:

FIG. 1 is a schematic sectional view of a lipstick applicator according to the invention; and

FIG. 2 is a schematic sectional view of another lipstick applicator embodiment having a light conducting rod in the lipstick mass.

### DETAILED DESCRIPTION OF THE CURRENTLY PREFERRED EMBODIMENTS

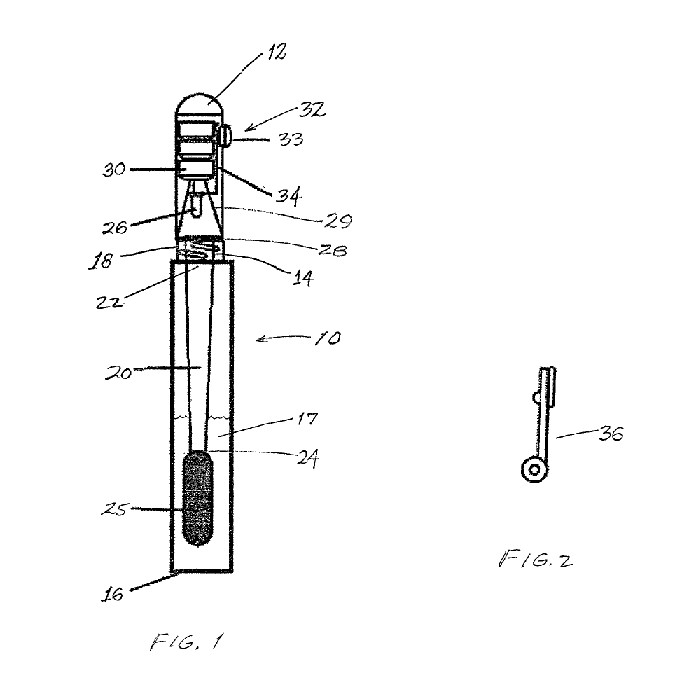

Referring initially to FIG. 1, the applicator 10 includes a first housing part 12 having a shoulder 14 which receives the open end of a second housing part 16 in a snap fit or, alternatively, threaded (not shown) engagement. The housing part 10 includes a sleeve 18 which receives a mass of light diffusing material 20 in the form of a bullet, the bullet having a fixed end 22 mounted in the sleeve 18 and a free end 24 remote from the first housing part 12. The bullet material may for example be one suitable for application to a user's lips, such as lipstick or lip gloss material. The material 20 may also be arranged in other shapes or forms in accordance with the particular material and/or its intended use of application.

A light source in the preferred form of a light emitting diode (LED) 26 is mounted in the housing part 12 adjacent to the fixed end 22 of the bullet, together with a collector plate 28 for focussing light from the LED onto the fixed end of the bullet. The LED 26 is powered for illumination by one or more button-type or watch batteries 30. The batteries are selectively connected in series with the LED 26 via user-actuation of a switch 32 which is accessible on the outside of the first housing part 12. Although the switch is shown in the drawings as being mounted on the side wall of the housing part 12, it may alternately be mounted in the end wall 33 or, indeed, at any desired location in the housing as a general matter of design choice. It will also be recognized that although the electrical wiring and/or contents or current flow paths are not shown in the drawings, they may be implanted in any desired or appropriate manner to provide the intended and herein disclosed functionality.

FIG. 2 depicts another embodiment of an applicator 10 in accordance with the invention, and which employs several features that may be substituted for or added to the applicator 10 shown in FIG. 1. In the applicator 10′ a light conduction rod 34 extends into the bullet 20 of light diffusing material 20 from the fixed end 22, for transmitting light from the LED 26 within and along the bullet toward the fixed end 24. Radial projections 36 transmit light toward predetermined locations on or proximate the peripheral surface of the bullet to provide a desired light pattern. In order to facilitate and enhance the transmission of LED-generated light into the light conduction rod 34, a lens 38 and a reflector 40 are provided between the LED 26 and bullet end 22, though the applicator may alternatively be implemented so that either of these features alone provides adequate focussing. It is also contemplated that the end of the rod 34 adjacent the fixed bullet end 22 may be provided with an integral portion, such as a disk, that further assists in gathering and transmitting light from the LED into the rod 34.

An electronics package 42 shown at the bottom of the first housing part 12 may implement any number of associated operating functions including, by way of example, flashing or intermittently powering the LED, or sensing light and/or motion for use in activating the LED, or maintaining the LED in an illuminated condition for a predetermined or random period of time after the switch is pressed, or some combination of these and/or other functions. The design and connection of the package 42 to the remainder of the components will be apparent to persons of skill in the art.

While there have shown and described and pointed out fundamental novel features of the invention as applied to preferred embodiments thereof, it will be understood that various omissions and substitutions and changes in the form and details of the devices illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

I claim:

1. An applicator for applying material to a surface, said applicator comprising
a first housing part,
a mass of light diffusing material having a fixed end mounted in said first housing part and projecting from said fixed end to a free end remote from said housing part, said material being selectively applyable to a surface by contact with said surface, and
a light source mounted in said first housing part so that light emitted from the light source enters said fixed end of said mass and is transmitted into and at least partially through and along said mass.

2. An applicator as in claim 1, further comprising a second housing part which mates with said first housing part to enclose said mass of light diffusing material within the mated first and second housing parts.

3. An applicator as in claim 2, wherein said second housing part is made of a light transmissive material.

4. An applicator as in claim 1, wherein said mass of light diffusing material is in the shape of a bullet.

5. An applicator as in claim 1, wherein said light source comprises an LED.

6. An applicator as in claim 1, further comprising means for focussing light emitted by said light source onto said fixed end of said mass.

7. An applicator as in claim 6, wherein said means for focussing light comprises a lens.

8. An applicator as in claim 6, wherein said means for focussing light comprises a reflector.

9. An applicator as in claim 1, wherein said mass of light diffusing material is translucent.

10. An applicator as in claim 1, wherein said mass of light diffusing material comprises a light transmissive material containing reflective particles.

11. An applicator as in claim 1, further comprising at least one button-type battery for powering said light source.

12. An applicator as in claim 1, further comprising an elongated rod of light conducting material extending into said mass from said fixed end of the mass.

13. An applicator as in claim 12, further comprising a plurality light conducting projections extending radially from said elongated rod of light conducting material.

14. An applicator as in claim 1, further comprising means for selectively activating said light source mounted in said first housing part.

15. An applicator as in claim 1, wherein said mass of light diffusing material comprises a light transmissive material containing at least one of phosphorescent and flourescent particles, and said light source emitting ultraviolet light.
